(19) United States
(12) Patent Application Publication
(10) Pub. No.: US 2022/0095954 A1
(43) Pub. Date: Mar. 31, 2022

(54) A FOOT MOUNTED WEARABLE DEVICE AND A METHOD TO OPERATE THE SAME

(71) Applicant: GT SILICON PRIVATE LIMITED, KANPUR (IN)

(72) Inventors: AMIT KUMAR GUPTA, KANPUR (IN); AJIT GUPTA, KANPUR (IN); SUBHOJYOTI BOSE, BANGALORE (IN)

(21) Appl. No.: 17/425,388

(22) PCT Filed: Feb. 7, 2020
(86) PCT No.: PCT/IB2020/050972

§ 371 Date: Jul. 23, 2021

(30) Foreign Application Priority Data
Feb. 8, 2019 (IN) 201911005132

(51) Int. Cl. A61B 5/11; A61B 5/00; G16H 20/30

(52) U.S. Cl. CPC A61B 5/112; A61B 5/1123; G16H 20/30; A61B 5/1112; A61B 5/6829; A61B 5/1118

(57) ABSTRACT

A foot mounted wearable device is disclosed. The device includes a toe-ring which includes a plurality of sensors configured to sense motion data associated with a foot of a user. The toe-ring also includes a microcontroller which includes a motion information processing subsystem configured to process the sensed motion data acquired from the plurality of sensors to determine motion information associated with the user. The device also includes a motion information analysis subsystem configured to receive the motion information associated with the user via an established communication network. The motion information analysis subsystem is also configured to analyse received motion information associated with the user to derive knowledge of at least one function associated with the user, wherein the at least one function includes at least one of gesture recognition, steps tracking of the user or a combination thereof.

### CROSS-REFERENCE TO RELATED APPLICATIONS

This application claims priority from a provisional patent application filed in India bearing no. 201911005132 titled “SMART TOE-RINGS FOR CAPTURING MOVEMENTS AND GESTURES” filed on Feb. 8, 2019.

### BACKGROUND

Embodiments of a present disclosure relate to a compact and comfortable wearable electronic device and more particularly to a foot mounted wearable device and a method to operate the same.

Wearable devices or wearables are electronic devices which are incorporated into clothing or worn on body of an individual as implants or accessories. The wearable devices are used for tracking information on real time basis. The wearable devices have motion sensors that take the snapshot of our day to day activity and sync them with mobile devices or computers. The wearable devices presently available in the market are of one or more types which includes smart watch, fitness tracker, head mounted display, foot mounted wearable device and the like. The foot mounted wearable device amongst the one or more wearable electronic devices is used for capturing precise motion information related to human movements, gait and gesture, for indoor navigation or gesture capturing or gait analysis, are either in the form of insole or are an externally attachable compact device. Various types of foot mounted wearable devices are available which helps in tracking the foot movement of the user.

Conventionally, the foot mounted wearable devices available in the market are worn by the user in the foot or such devices are mounted with shoes of the user. However, such devices cause hindrance in the foot movement and sometimes become inconvenient to use by the user. Also, such conventional foot mounted wearable devices are less durable, and performance of the device depends upon the quality of mounting. Moreover, shaking or sliding of one or more sensors of the foot mounted wearable device during foot movement, results in performance deterioration due to improper capture of motion data and compromises accuracy.

Hence, there is a need for an improved foot mounted wearable device and a method to operate the same in order to address the aforementioned issues.

### BRIEF DESCRIPTION

In accordance with an embodiment of the present disclosure, a foot mounted wearable device is disclosed. The foot mounted wearable device includes at least one toe-ring. The at least one toe-ring includes a plurality of sensors configured to sense motion data associated with a foot of a user. The at least one toe-ring also includes a microcontroller operatively coupled to the plurality of sensors. The microcontroller includes a motion information processing subsystem configured to process the sensed motion data acquired from the plurality of sensors to determine motion information associated with the user. The device also includes a motion information analysis subsystem hosted on a server and operatively coupled to the microcontroller. The motion information analysis subsystem is configured to receive the motion information associated with the user via an established communication network. The motion information analysis subsystem is also configured to analyse received motion information associated with the user to derive knowledge of at least one function associated with the user based on computation of a plurality of gait parameters and motion equation, wherein the at least one function associated with the user includes at least one of gesture recognition of the user, steps tracking of the user or a combination thereof.

In accordance with another embodiment of the present disclosure, a method of operation of a foot mounted wearable device is disclosed. The method includes sensing, by a plurality of sensors of at least one toe-ring, motion data associated with a foot of a user. The method also includes processing, by a motion information processing subsystem of a microcontroller, the sensed motion data acquired from the plurality of sensors to determine motion information associated with the user. The method also includes receiving, by a motion information analysis system hosted on a server, the motion information associated with the user via an established communication network. The method also includes analysing, by the motion information analysis subsystem, received motion information associated with the user to derive knowledge of at least one function associated with the user based on computation of a plurality of gait parameters and motion equation, wherein the at least one function associated with the user includes at least one of gesture recognition of the user, steps tracking of the user or a combination thereof.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram of a foot mounted wearable device in accordance with an embodiment of the present disclosure;

FIG. 2 depicts a schematic representation of top view of triple toe-rings connected by a chain in accordance with an embodiment of present disclosure;

FIG. 3 depicts a schematic representation of top view of a toe-rings with sensors and one or more components in accordance with an embodiment of present disclosure;

FIG. 4 represents a schematic representation of one embodiment of a foot mounted wearable device of FIG. 1 in accordance with an embodiment of the present disclosure;

FIG. 5 illustrates a schematic representation of an exemplary embodiment of a foot mounted wearable device in accordance with an embodiment of the present disclosure;

FIG. 6 depicts a schematic representation of an embodiment to represent a process of outdoor positioning solution using a foot mounted wearable device of FIG. 1 in accordance with an embodiment of the present disclosure;

FIG. 7 depicts a schematic representation of an embodiment to represent a process of indoor positioning solution using a foot mounted wearable device of FIG. 1 in accordance with an embodiment of the present disclosure;

FIG. 8 depicts a schematic representation of an embodiment of land surveying for a user using a foot mounted wearable device of FIG. 1 in accordance with an embodiment of the present disclosure;

FIG. 9 depicts a schematic representation of an embodiment of tracking physical exercise using a foot mounted wearable device of FIG. 1 in accordance with an embodiment of the present disclosure;

FIG. 10 is a block diagram of a computer or a server in accordance with an embodiment of the present disclosure; and

FIG. 11 is a flow chart representing the steps involved in a method of operation of a foot mounted wearable device of FIG. 1 in accordance with the embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a foot mounted wearable device and a method to operate the same. The device includes at least one toe-ring, wherein the at least one toe-ring includes a plurality of sensors configured to sense motion data associated with a foot of a user. The at least one toe-ring also includes a microcontroller operatively coupled to the plurality of sensors. The microcontroller includes a motion information processing subsystem configured to process the sensed motion data acquired from the plurality of sensors to determine motion information associated with the user. The device also includes a motion information analysis subsystem hosted on a server and operatively coupled to the microcontroller. The motion information analysis subsystem is configured to receive the motion information associated with the user via an established communication network. The motion information analysis subsystem is also configured to analyse received motion information associated with the user to derive knowledge of at least one function associated with the user based on computation of a plurality of gait parameters and motion equation the at least one function associated with the user includes at least one of gesture recognition of the user steps tracking of the user or a combination thereof.

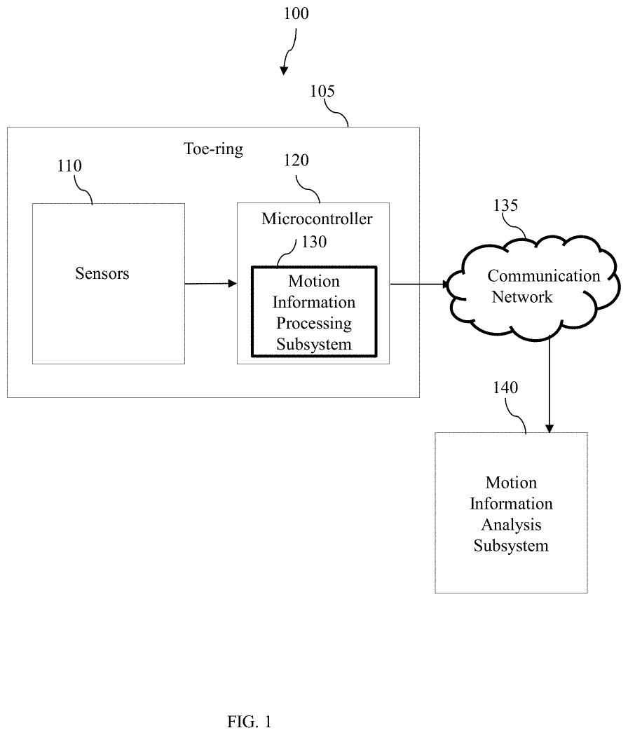

FIG. 1 is a block diagram of a foot mounted wearable device 100 in accordance with an embodiment of the present disclosure. The foot-mounted wearable device includes at least one toe-ring worn by the user on the toes. As used herein, the term ‘foot mounted wearable device’ is defined as a wearable device with a flat base which is worn by a user in foot or toes without causing any hindrance in feet movement. In one embodiment, the at least one toe-ring may include an outer ring of a predefined diameter based on a user preference. In such embodiment, the at least one toe-ring may be attached to a compact plastic material or metal capsule to house a plurality of components based on user' convenience. In one embodiment, the toe-ring may completely encircle the toe of the user. In another embodiment, the toe-ring may include two open ends which may not meet in order to encircle the toe of the user. In yet another embodiment, the at least one toe-ring may include a flexible band-aid like structure which may be wrapped around the toe of the user. The at least one toe-ring 105 may include a set of one and more toe rings connected by flexible wires embedded inside a chain.

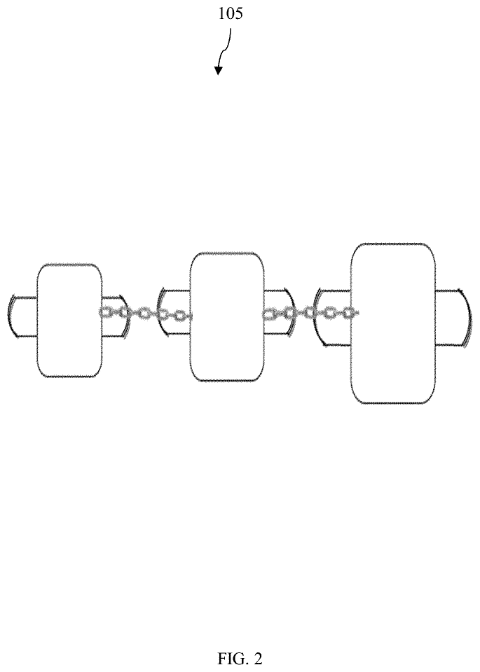

One such embodiment of the set of toe-rings connected by a chain is depicted in FIG. 2. FIG. 2 depicts a schematic representation of top view of triple toe-rings connected by a chain in accordance with an embodiment of present disclosure.

The set of toe-rings 105 (FIG. 1) worn together by the user provides a facility of smaller and thinner dimensions without losing any component or functionality and flexibility. An arrangement of the set of toe rings makes up more space for adding more sensors and components thus increasing the capability and applications of the device. In one embodiment, shape of the at least one toe-ring may include a square shape, a circular shape, a diamond shape, a round shape, an oval shape and the like.

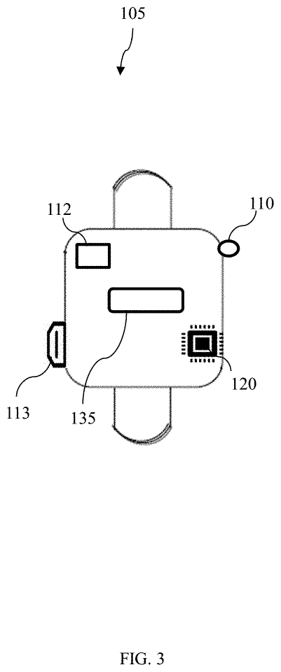

The at least one toe-ring 105 also includes a plurality of sensors 110 configured to sense motion data associated with a foot of a user. In one embodiment, the plurality of sensors 110 may include at least one of an accelerometer, a gyroscope, a pressure sensor, a magnetometer or a combination thereof. The at least one toe-ring 105 also includes a microcontroller 120 operatively coupled to the plurality of sensors 110. In a specific embodiment, the device 100 further includes a battery 112 to supply power for an operation at least one toe-ring 105 for performing multiple functions. In such embodiment, the battery may be recharged through a charging port 113. One embodiment of the at least one toe-ring with the plurality of sensors 110 and electronic components such as the battery 112, the charging port 113, the microcontroller (120 and the communication subsystem 135) to establish the communication network is depicted in FIG. 3. FIG. 3 depicts a schematic representation of top view of a toe-rings with sensors and one or more components in accordance with an embodiment of present disclosure.

In some embodiment, the at least one toe-ring may include a compact plastic or a metallic capsule which may include the plurality of sensors. In another embodiment, the plurality of sensors may be attached on a periphery of a housing of the at least one toe-ring. In one embodiment, the at least one toe-ring may house the plurality of sensors 110 and one or more components. In such embodiment, the at least one toe-ring may be fabricated from fibre, carbon fibre, selective laser sintering (SLS) material, rubber, flexible materials and the like. The microcontroller 120 includes a motion information processing subsystem 130 configured to process the sensed motion data acquired from the plurality of sensors 110 to determine motion information associated with the user. In one embodiment, the motion data acquired from the plurality of sensors 110 may include linear motion data associated with the foot of the user, rotational motion data associated with the foot of the user or a combination thereof. In some embodiment, the motion data acquired from the plurality of sensors 110 may include at least one of position of the foot, acceleration of the foot, velocity of the foot, orientation of the foot or a combination thereof. In some embodiment, the accelerometer and the gyroscope may be used to capture fast movement of feet of the user by sampling acceleration and angular velocity data with high frequency. In one embodiment, the orientation of the foot may be obtained using the magnetometer and or the gyroscope. In another embodiment, the pressor sensor may be utilised to obtain height from sea level.

In one embodiment, the motion information may include at least one of step count, step size, traversed path, gesture, foot movement or a combination thereof. The motion information processing subsystem 130 also removes electronic noises or errors from sensed motion data acquired from the plurality of users by using a noise removal technique. In such embodiment, the noise removal technique may include a zero-velocity update (ZUPT) based pedestrian dead reckoning (PDR) technique. In a specific embodiment, the motion information processing subsystem 130 monitors positional accuracy of the user based on a positioning system, wherein the positioning system includes an indoor positioning system (IPS) and/or an outdoor positioning system.

A position obtained from the plurality of sensors 110 are fused with positioning obtained from an outdoor positioning receiver. In one embodiment, the outdoor positioning receiver may include a global positioning system (GPS) receiver. Such process is particularly useful in urban environment where building structures obstruct direct GPS satellite signals every now and then. Similarly, one possible way of realizing indoor positioning and navigation is by deploying one or more beacon systems which includes a wireless technology such as wireless fidelity (Wi-Fi) technology or Bluetooth technology. But such indoor positioning systems also suffer from inaccuracies. Here also, the Wi-Fi/Bluetooth technology maybe fused to the plurality of sensors to get the indoor navigation system (INS) enhancing positional accuracy. In one embodiment, data obtained from the positioning system is sent through a communication subsystem to a user interface of electronic device associated with a user. In another embodiment, data obtained from the positioning system is sent to a remote server through the communication subsystem (not shown in FIG. 1). In such embodiment, the communication subsystem is configured to establish the wireless communication network 135 with one or more user devices for transmission of received motion information to the remote server through at least one of a Bluetooth low energy (BLE) technology, wireless fidelity (Wi-Fi) networking technology, near field communication (NFC), long term evolution (LTE) communication standard or a combination thereof.

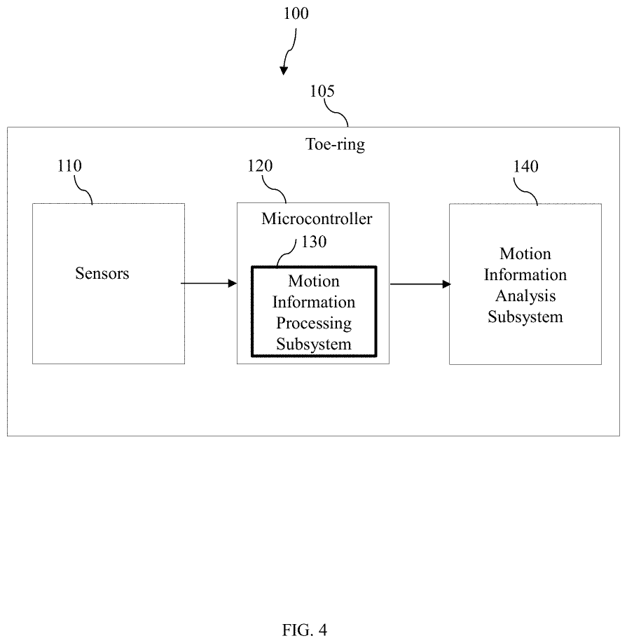

The device 100 also includes a motion information analysis subsystem 140 hosted on a server and operatively coupled to the microcontroller 120. In one embodiment, the motion information analysis subsystem 140 may be located on the at least one toe-ring 105. One such embodiment of the toe-ring with the motion information analysis subsystem 140 located locally on the toe-ring 105 is depicted in FIG. 4. FIG. 4 represents a schematic representation of one embodiment of a foot mounted wearable device 100 of FIG. 1 in accordance with an embodiment of the present disclosure. In another embodiment, the motion information analysis subsystem 140 may be hosted on the remote server such as the cloud server. In such embodiment, the remote server may include a cloud server. The motion information analysis subsystem 140 is configured to receive the motion information associated with the user via an established communication network 135. The motion information analysis subsystem 140 is also configured to analyse received motion information associated with the user to derive knowledge of at least one function associated with the user based on computation of a plurality of gait parameters and motion equation, wherein the knowledge of the at least one function associated with the user may include at least one of gesture recognition of the user, tracking steps of the user or a combination thereof. The gesture recognition of the user is further utilised for fitness monitoring of the user, gait analysis of the user and diagnosing movement disorder of the user. Again, the tracking steps of the user is further utilised for tracking the user, outdoor positioning of the user, indoor positioning of the user, land surveying for the user and the like. In some embodiment, diagnosing the movement disorder of the user may include a preliminary diagnosis using the foot mounted device worn by the user. In such embodiment, the plurality of gait parameters are computed by sampling the motion data obtained from the plurality of sensors corresponding to both left foot and right foot of the user. In such embodiment, the plurality of gait parameters may include at least one of cadence, gait cycle, stance phase, swing phase, average, speed, step length, step time, single support, double support or a combination thereof. Furthermore, the present disclosed device also enables steps tracking of the user which further helps in pedestrian positioning in absence of GPS, enhancing performance of the GPS based positioning system, by fusing positioning data obtained using toe-ring, enhancing performance of the indoor positioning system by fusing positioning data obtained using toe-ring, land survey (or measurement of land parcel) by tracking steps and the like.

In one embodiment, the knowledge associated with the land survey for the user may be derived based on computation of displacement and heading change at every step from the inertial position data of the foot mounted device. In some embodiment, the motion information analysis subsystem 140 analyses the inertial position data of the user and constructs a user's resultant path. Here, the inertial data or step's coordinates are stored in a log file for future reference. Such stored data are later utilised in conducting land survey of urban slums or the congested areas where the open sky is not easily accessible to allow GPS signals to reach to ground, narrow crowded lanes, which are curved or crooked, and are difficult to survey using traditional method of using total station and sensitive areas where land survey may not be carried out openly due to security reasons.

In yet another embodiment, the knowledge of the fitness monitoring associated with the user may be derived by identifying one or more body gestures. The one or more body gestures of the user which are recognised further helps in fitness monitoring, sports analytics, gesture-based commands. The motion information analysis subsystem 140 derives knowledge of the fitness monitoring for the user by detecting a type of one or more exercises and counting number of the one or more exercises performed by the user based on the received motion information. In one embodiment, the type of the one or more exercises may include at least one of squats, crunches, halasana, jumping jacks and the like. The type of the one or more exercises performed by the user are detected from raw data obtained from the plurality of sensors. Such collected raw data is analysed by a sensor fusion stack (SFS) based on signal processing and machine learning techniques. In one embodiment, the SFS may enable implementation of heuristic model based on a fusion of deductive and heuristic techniques. In one embodiment, the SFS may be implemented on the foot mounted wearable device. In another embodiment, the SFS may be implemented on a user device associated with user. In yet another embodiment, the SFS may be implemented on the remote server such as the cloud server.

In a preferred embodiment, the device 100 further includes a notification subsystem 150 operatively coupled to the motion information analysis subsystem 140. The notification subsystem 150 is configured to notify a plurality of monitoring status upon analysis of the motion information to the user through a plurality of notification means. In one embodiment, the monitoring status may include at least one of the type of the one or more exercises, number of calories burnt, time interval of a particular exercise, and the like.

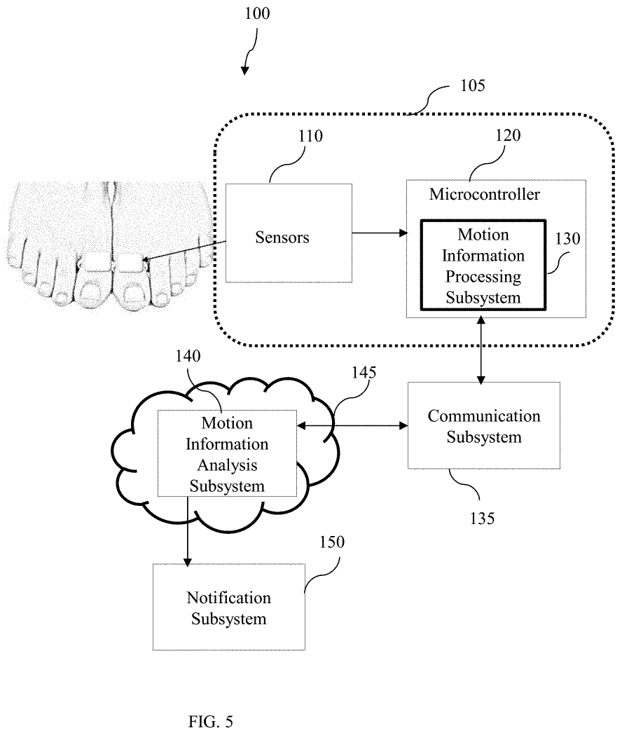

FIG. 5 illustrates a schematic representation of an exemplary embodiment of a foot mounted wearable device 100 in accordance with an embodiment of the present disclosure. The foot mounted wearable device 100 is worn by a user to obtain accurate movement data. Let us assume, that a user is a health-conscious person and wants to keep track of health records of herself or himself periodically. In such a case, the user may wear the foot-mounted wearable device which not only obtains accurate movement data but also keeps track of one or more physical activities performed by the user. Moreover, such foot-mounted wearable devices are comfortable to wear and also flexible to use. The user as a foot-mounted device 100 may use at least one toe-ring 105 which is worn by the user on toes, wherein such toe-ring helps in navigation, analysis of foot movement for preliminary diagnosis of movement disorder and capturing human gestures for fitness monitoring or any other purpose. Here, the at least one toe-ring 105 worn by the user may be of one or more shapes such as square, rectangular, circular and the like. Also, the toe-ring may be available in different sizes based on requirement of the user. The user may also wear more than one toe-ring based on requirement as an arrangement of the more than one toe ring makes up more space for adding more sensors and components thus increasing the capability and applications of the device. Here, the set of the toe-rings may be connected using a flexible chain like structure which helps the toe-ring keeping intact on the toes of the user.

The toe-ring 105 includes a plurality of sensors 110 configured to sense motion data associated with a foot of the user. Here, the motion data may include at least one of position of the foot, acceleration of the foot, velocity of the foot, orientation of the foot or a combination thereof. For example, the plurality of sensors may include at least one of an accelerometer, a gyroscope, a pressure sensor, a magnetometer or a combination thereof. Here, inertial sensors such as the accelerometer and the gyroscope may be used to capture fast movement of feet of the user by sampling acceleration and angular velocity data with high frequency. Again, the orientation of the foot may be obtained using the magnetometer and or the gyroscope. Similarly, the pressor sensor may be utilised to obtain height from sea level.

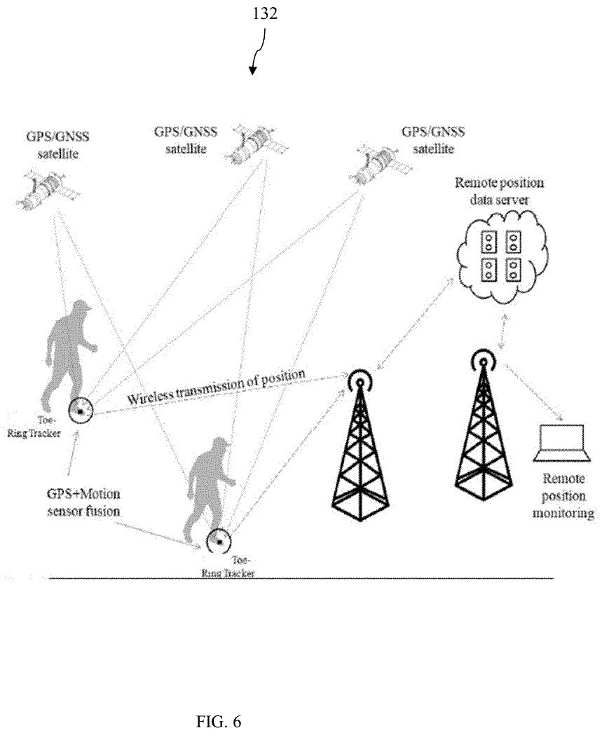

The toe-ring 105 also includes a microcontroller 120 wherein the microcontroller 120 includes a motion information processing subsystem 130 to process the sensed motion data acquired from the plurality of sensors to determine motion information associated with the user. Here, the motion data may be processed to obtain the motion information such as at least one of step count, step size, traversed path, gesture, foot movement or a combination thereof. The motion information processing subsystem 130 also removes electronic noises or errors from sensed motion data acquired from the plurality of users by using a noise removal technique. For example, the noise removal technique may include a zero-velocity update (ZUPT) based pedestrian dead reckoning (PDR) technique. Also, the motion information processing subsystem 130 monitors positional accuracy of the user based on positioning system, wherein the positioning system includes an indoor positioning system (IPS) and/or an outdoor positioning system. Here, the outdoor positioning system may include a global positioning system (GPS) to obtain the outdoor position. One such example of outdoor positioning solution using the device 100 is shown in FIG. 6. FIG. 6 depicts a schematic representation of an embodiment 132 to represent a process of outdoor positioning solution using a foot mounted wearable device of FIG. 1 in accordance with an embodiment of the present disclosure.

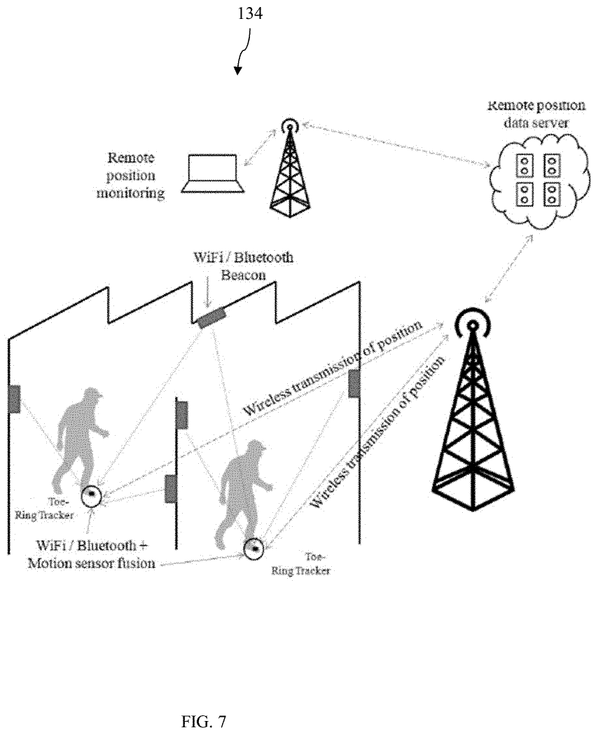

Referring back to FIG. 5, position obtained from the plurality of sensors 110 are fused with positioning obtained from an outdoor positioning receiver. In one embodiment, the outdoor positioning receiver may include a global positioning system (GPS) receiver. Such process is particularly useful in urban environment where building structures obstruct direct GPS satellite signals every now and then. Similarly, one possible way of realizing indoor positioning and navigation is by deploying one or more beacon systems which includes a wireless technology such as wireless fidelity (Wi-Fi) technology or Bluetooth technology. But such indoor positioning systems also suffer from inaccuracies. Here also, the Wi-Fi/Bluetooth technology maybe fused to the plurality of sensors to get the indoor navigation system (INS) enhancing positional accuracy. So, the positioning system which fuses the two technologies such as the IPS and/or the GPS enhances positional accuracy. In one embodiment, data obtained from the positioning system is sent through a communication subsystem to a user interface of electronic device associated with a user. Similarly, an example of an indoor positioning solution using the foot mounted wearable device is shown in FIG. 7. FIG. 7 depicts a schematic representation of an embodiment 134 to represent a process of indoor positioning solution using a foot mounted wearable device of FIG. 1 in accordance with an embodiment of the present disclosure.

Again, referring back to FIG. 5, the device 100 also includes a motion information analysis subsystem 140 hosted on a cloud server 145 to receive the motion information associated with the user via an established communication network. Upon, receiving the motion information, the motion information analysis subsystem 140 analyses received motion information associated with the user to derive knowledge of at least one function associated with the user based on computation of a plurality of gait parameters and motion equation. For example, the knowledge of the at least one function associated with the user may include at least one of gesture recognition of the user, steps tracking of the user or a combination thereof. The gesture recognition is utilised in diagnosing movement disorder of the user, gait analysis of the user, fitness monitoring of the user and the like. Similarly, the steps tracking of the user is utilised in tracking the user, outdoor positioning of the user, indoor positioning of the user, land surveying for the user and the like.

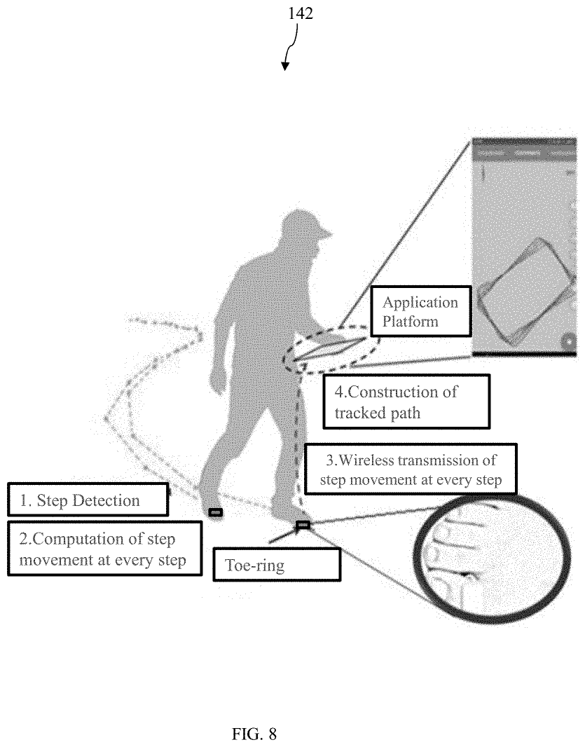

Here, the knowledge associated with the land survey for the user may be derived based on computation of displacement and heading change at every step from the inertial position data of the foot mounted device. For example, the motion information analysis subsystem analyses the inertial position data of the user and constructs a user's resultant path which is further notified on the electronic device associated with user. One such representation of the land survey process using the foot mounted wearable device is shown in FIG. 8. FIG. 8 depicts a schematic representation of an embodiment 142 of land surveying for a user using a foot mounted wearable device of FIG. 1 in accordance with an embodiment of the present disclosure.

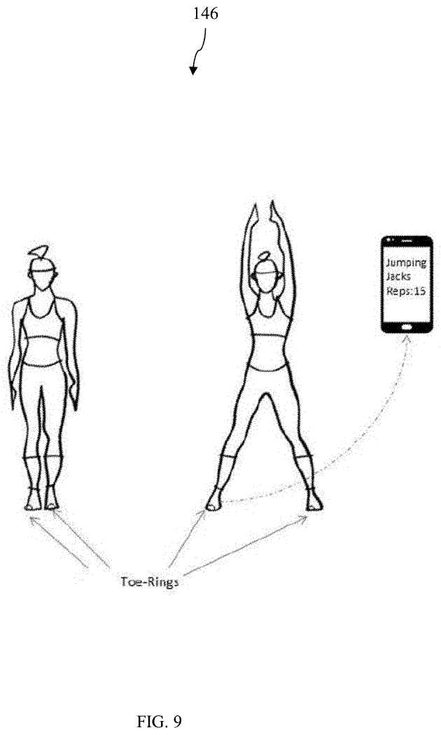

Similarly, the knowledge associated with the fitness monitoring of the user may include tracking physical workouts or one or more exercises performed by the user. Here, the motion information analysis subsystem 140 identifies gestures of the user to detect a type of the one or more exercises and counting number of the one or more exercises performed by the user based on the received motion information. For example, the type of the one or more exercises may include at least one of squats, crunches, halasana, jumping jacks and the like. One such example of the jumping jacks performed by the user is shown in FIG. 9. FIG. 9 depicts a schematic representation of an embodiment 146 of tracking physical exercise using a foot mounted wearable device of FIG. 1 in accordance with an embodiment of the present disclosure.

The motion information analysis subsystem 140 (FIG. 5) hosted on the cloud server 145 detects multiple variants of a same exercise and keeps track of a particular variant count as depicted in FIG. 9. Once, the particular variant count reaches a predefined threshold value, then such detection process is rejected and runs for detection of next variant of the one or more exercises.

Further, the device 100 also includes a notification subsystem 150 to notify a plurality of monitoring status upon analysis of the motion information to the user through a plurality of notification means. Here, the monitoring status may include at least one of the type of the one or more exercises, number of calories burnt, time interval of a particular exercise, and the like. Thus, the device 100 helps the user in overall manner by tracking the feet movement, monitoring the movement disorders, monitoring health parameters and alerting the user to create awareness.

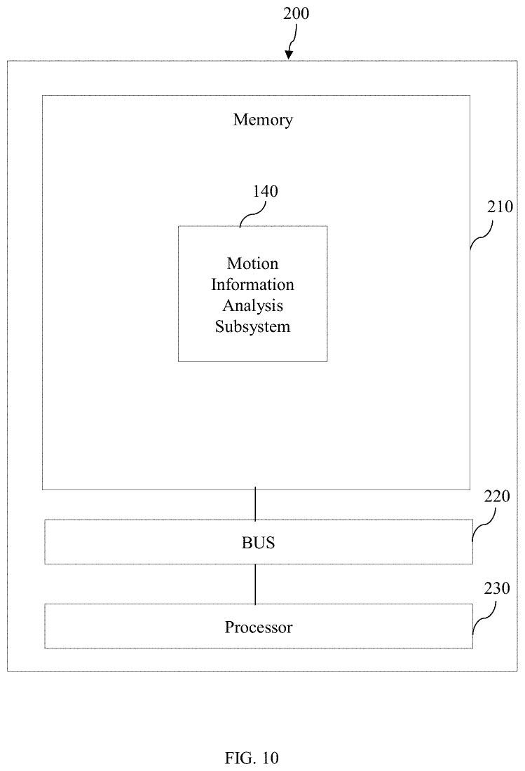

FIG. 10 is a block diagram of a computer or a server in accordance with an embodiment of the present disclosure. The server 200 includes processor(s) 230, and memory 210 operatively coupled to the bus 220.

The processor(s) 230, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 210 includes a subsystem stored in the form of executable program which instructs the processor 230 to perform the method steps illustrated in FIG. 1. The memory 210 has following subsystem: a motion information analysis subsystem 140.

The motion information analysis subsystem 140 is configured to receive motion information associated with the user via an established communication network. The motion information analysis subsystem is also configured to analyse received motion information associated with the user to derive knowledge of at least one function associated with the user based on computation of a plurality of gait parameters and motion equation, wherein the at least one function associated with the user includes at least one of gesture recognition of the user, tracking steps of the user or a combination thereof.

The bus 220 as used herein refers to be internal memory channels or computer network that is used to connect computer components and transfer data between them. The bus 220 includes a serial bus or a parallel bus, wherein the serial bus transmit data in bit-serial format and the parallel bus transmit data across multiple wires. The bus 220 as used herein, may include but not limited to, a system bus, an internal bus, an external bus, an expansion bus, a frontside bus, a backside bus and the like.

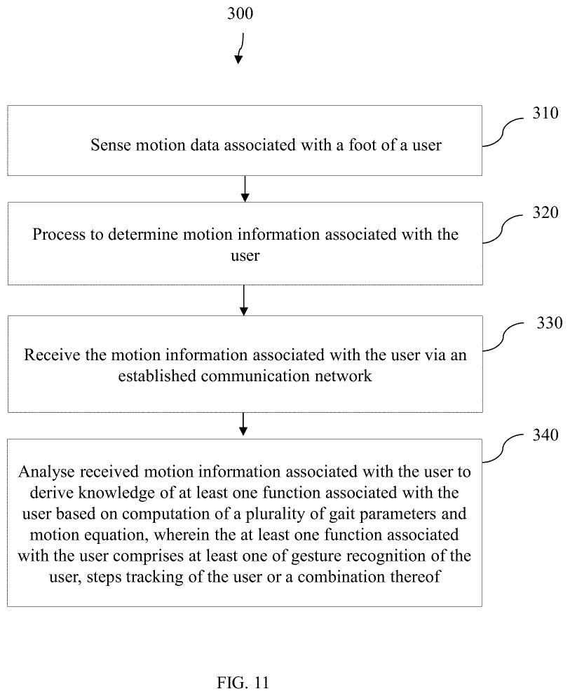

FIG. 11 is a flow chart representing the steps involved in a method 300 of operation of a foot mounted wearable device of FIG. 1 in accordance with the embodiment of the present disclosure. The method 300 includes sensing, by a plurality of sensors of a toe-ring, motion data associated with a foot movement of a user in step 310. In one embodiment, sensing the motion data associated with the foot movement of the user may include sensing at least one of position of the foot, acceleration of the foot, velocity of the foot, orientation of the foot or a combination thereof. In such embodiment, sensing the motion data associated with the foot movement of the user may include sensing the motion data through the plurality of sensors, wherein the plurality of sensors my include but not limited to, an accelerometer, a gyroscope, a pressure sensor, a magnetometer or a combination thereof.

The method 300 also includes processing, by a motion information processing subsystem of a microcontroller of the toe-ring, the sensed motion data acquired from the plurality of sensors to determine motion information associated with the user in step 320. In one embodiment, processing the sensed motion data to obtain the motion information may include processing the sensed motion data to obtain information of at least one of step count, step size, traversed path, gesture, foot movement or a combination thereof. In some embodiment, the motion data may be processed by removing electronic noises or errors from sensed motion data acquired from the plurality of users by using a noise removal technique. In such embodiment, the noise removal technique may include a zero-velocity update (ZUPT) based pedestrian dead reckoning (PDR) technique.

The method 300 also includes receiving, by a motion information analysis system hosted on a server, the motion information associated with the user via an established communication network in step 330. In one embodiment, receiving the motion information associated with the user via the established network may include receiving the motion information via a wireless communication network established with one or more user devices for transmission of received motion information to the remote server through at least one of a Bluetooth low energy (BLE) technology, wireless fidelity (Wi-Fi) networking technology, near field communication (NFC), long term evolution (LTE) communication standard or a combination thereof.

The method 300 also includes analysing, by the motion information analysis subsystem, received motion information associated with the user to derive knowledge of at least one function associated with the user based on computation of a plurality of gait parameters and motion equation in step 340. In one embodiment, analysing the received motion information associated with the user to derive the knowledge of the at least one function may include analysing the received motion information to derive knowledge of at least one of gesture recognition of the user, steps tracking of the user or a combination thereof.

Various embodiments of the present disclosure relate to a foot-mounted wearable device which helps to obtain accurate movement data when the tracking sensor is worn as a toe-ring.

Moreover, the present disclosed system helps in preliminary diagnosis of movement disorders associated with the user based on gait analysis through the toe-ring which generally makes the user aware and motivates him or her for further consultation with a medical practitioner.

Also, the present disclosed device enables gesture recognition of the user which further helps in fitness monitoring, sports analytics, gesture-based commands.

Furthermore, the present disclosed device also enables steps tracking of the user which further helps in pedestrian positioning in absence of GPS, enhancing performance of the GPS based positioning system, by fusing positioning data obtained using toe-ring, enhancing performance of the indoor positioning system by fusing positioning data obtained using toe-ring, land survey (or measurement of land parcel) by tracking steps and the like.

Moreover, the indoor positioning technology has applications in tracking rescue agents (firefighters, mining rescue agents etc) when they perform rescue operation indoor. As a result, real-time indoor position which is obtained from indoor pedestrian positioning technology helps in case of emergency and may be used for tracking workforce, for better management and safely, in airports, factories, hotels etc.

Again, the present disclosed device provides more accurate results in capturing the motion information as sometimes conventional wearable devices which are worn on wrist or hand of the user may consider a footstep count even if there is some sort of hand movement.

In addition to, the present disclosed device is also capable of capturing hand movements of the user when worn by the user in fingers while performing the one or more physical exercises and thus helps in identifying the type of the one or more physical exercises, wherein the hand movement is captured by using one or more inertial sensors and further such captured hand movements are analysed and recognised by using a signal processing and machine learning technique.

It will be understood by those skilled in the art that the foregoing general description and the following detailed description are exemplary and explanatory of the disclosure and are not intended to be restrictive thereof.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A foot mounted wearable device comprising:
at least one toe-ring, wherein the at least one toe-ring comprises:
a plurality of sensors configured to sense motion data associated with a foot of a user;
a microcontroller operatively coupled to the plurality of sensors, wherein the microcontroller comprises a motion information processing subsystem configured to process the sensed motion data acquired from the plurality of sensors to determine motion information associated with the user;
a motion information analysis subsystem hosted on a server and operatively coupled to the microcontroller, wherein the motion information analysis subsystem is configured to:
receive the motion information associated with the user via an established communication network; and
analyse received motion information associated with the user to derive knowledge of at least one function associated with the user based on computation of a plurality of gait parameters and motion equation, wherein the at least one function associated with the user comprises at least one of gesture recognition of the user, steps tracking of the use or a combination thereof.

2. The device as claimed in claim 1, wherein the plurality of sensors comprises at least one of an accelerometer, a gyroscope, a pressure sensor, a magnetometer or a combination thereof.

3. The device as claimed in claim 1, wherein the motion data comprises linear motion data associated with the foot of the user or rotational motion data associated with the foot of the user.

4. The device as claimed in claim 1, wherein the motion information comprises at least one of step count, step size, traversed path, foot movement or a combination thereof.

5. The device as claimed in claim 1, wherein the motion information processing subsystem is configured to monitor positional accuracy of the user based on positioning system, wherein the positioning system comprises at least one of an indoor positioning system (IPS) an outdoor positioning system or a combination thereof.

6. The device as claimed in claim 1, wherein the plurality of gait parameters comprises at least one of cadence, gait cycle, stance phase, swing phase, average, speed, step length, step time, single support, double support or a combination thereof.

7. The device as claimed in claim 1, wherein the motion information analysis subsystem is configured to derive knowledge of the at least one function associated with the user for diagnosing movement disorder of the user, fitness monitoring of the user and land survey for the user.

8. The device as claimed in claim 1, wherein the motion information analysis subsystem is configured to derive knowledge of the fitness monitoring for the user by detecting a type of one or more exercises and counting number of the one or more exercises performed by the user based on the received motion information.

9. The device as claimed in claim 1, further comprising a notification subsystem operatively coupled to the motion information analysis subsystem, wherein the notification subsystem is configured to notify a plurality of monitoring status upon analysis of the motion information to the user through a plurality of notification means.

10. A method comprising:
sensing, by a plurality of sensors of a toe-ring, motion data associated with a foot of a user;
processing, by a motion information processing subsystem of a microcontroller, the sensed motion data acquired from the plurality of sensors to determine motion information associated with the user;
receiving, by a motion information analysis system hosted on a server, the motion information associated with the user via an established communication network; and
analysing, by the motion information analysis subsystem hosted on the server, received motion information associated with the user to derive knowledge of at least one function associated with the user based on computation of a plurality of gait parameters and motion equation, wherein the at least one function associated with the user comprises at least one of gesture recognition of the user, steps tracking of the user or a combination thereof.
